(19) United States
(12) Patent Application Publication
(10) Pub. No.: US 2025/0359962 A1
(43) Pub. Date: Nov. 27, 2025

(54) TWEEZERS AND A METHOD OF CONDUCTING ROOT CANAL THERAPY

(71) Applicant: Kehn YAPP, Hornsby, New South Wales (AU)

(72) Inventor: Kehn YAPP, Hornsby, New South Wales (AU)

(21) Appl. No.: 18/874,818

(22) PCT Filed: Jun. 7, 2023
(86) PCT No.: PCT/AU2023/050498

§ 371 Date: Dec. 13, 2024

(30) Foreign Application Priority Data
Jun. 14, 2022 (AU) 2022901614

(51) Int. Cl. A61C 5/42

(52) U.S. Cl. CPC A61C 5/42

(57) ABSTRACT

Tweezers for use in conducting root canal therapy and methods of conducting root canal therapy using the tweezers are described, the tweezers including: a pair of elongate arms; the arms are moveable between a closed condition and an open condition and are biased towards the closed condition; a grasping formation is provided at the end of each of the arms; the grasping formations have substantially part cylindrical faces to grasp opposite sides of a substantially cylindrical item; and wherein the axes of the cylindrical faces are offset from a direction perpendicular to the length of the arms.

### TECHNICAL FIELD

The present invention relates to tweezers for use in conducting root canal therapy and to a method of conducting root canal therapy using tweezers.

### BACKGROUND TO THE INVENTION

When conducting root canal therapy it is necessary to remove material from the root canal of a tooth. This is usually done with the aid of one or more endodontic hand files of various types and diameters. The file is slender and is configured to ream out and otherwise remove matter from the root canal of a tooth.

A lockable pair of forceps are sometimes used to hold endodontic files during root canal therapy. These typically have a locking clasp to secure the forceps around the handle of the file. However, the locking clasp can sometimes loosen, unintentionally releasing the file. Furthermore, in some instances the locking clasp may be subject to jamming which prevents the forceps from engaging and disengaging the endodontic hand file.

There remains a need for improved measures to conduct root canal therapy.

### SUMMARY OF THE INVENTION

In a first aspect the present invention provides tweezers for use in conducting root canal therapy including: a pair of elongate arms; the arms are moveable between a closed condition and an open condition and are biased towards the closed condition; a grasping formation is provided at the end of each of the arms; the grasping formations have substantially part cylindrical faces to grasp opposite sides of a substantially cylindrical item; and wherein the axes of the cylindrical faces are offset from a direction perpendicular to the length of the arms.

The axes of the cylindrical faces may be offset by between 10 and 30 degrees.

The axes of the cylindrical faces may be offset by between 15 and 25 degrees.

The axes of the cylindrical faces may be offset by between 17 to 23 degrees.

In a second aspect the present invention provides a method of conducting root canal therapy including the steps of: providing tweezers, the tweezers including: a pair of elongate arms; the arms are moveable between a closed condition and an open condition and are biased towards the closed condition; a grasping formation is provided at the end of each arm; the grasping formations have substantially part cylindrical faces to grasp opposite sides of a substantially cylindrical item; the method further including the steps of: grasping a file using the tweezers; and using the tweezers and the file to conduct root canal therapy.

### BRIEF DESCRIPTION OF THE DRAWINGS

An embodiment of the present invention will now be described, by way of example only, with reference to the accompanying drawings, in which:

FIG. 1 is side view of a pair of tweezers for use in conducting root canal therapy;

FIG. 2 is a top view of the tweezers of FIG. 1 in the closed condition;

FIG. 3 is a top view of the tweezers of FIG. 1 in the open condition;

FIG. 4 is a perspective view showing the tweezers of FIG. 1 being grasped in a hand, and moving the tweezers from the closed to the open condition;

FIG. 5 is a perspective view showing the tweezers of figure grasping an endodontic hand file;

FIGS. 6A and 6B show the tweezers and file in two orientations with the file tip pointing outwardly; and

FIGS. 7A and 7B show the tweezers and file in two orientations with the file tip pointing inwardly.

### DETAILED DESCRIPTION OF THE PREFERRED EMBODIMENT

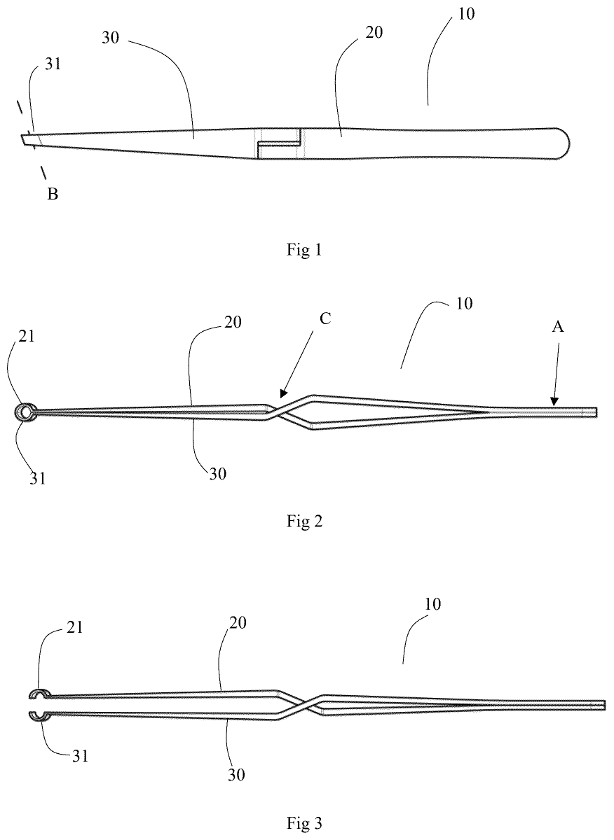

Referring to FIGS. 1 and 2, a pair of tweezers 10 for use in conducting root canal therapy is shown including: a pair of elongate steel arms 20, 30. The arms are fused together at region A. In contrast to regular tweezers, tweezers 10 are “reverse action” which is effected by the arms 20, 30 crossing over one another at region C.

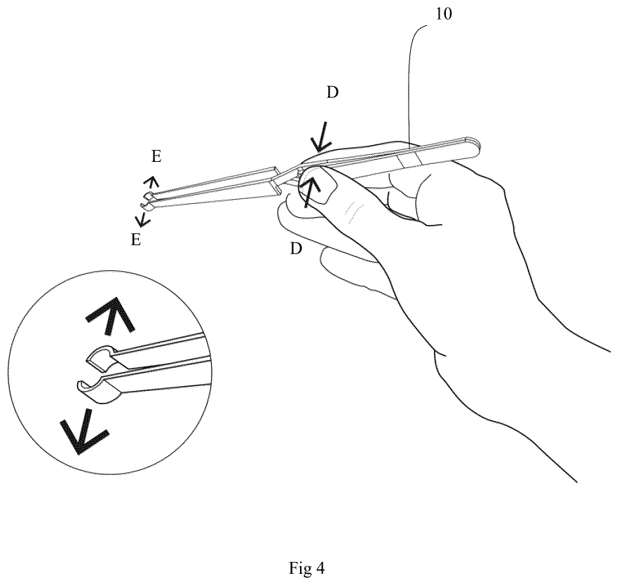

The arms are moveable between a closed condition as shown in FIG. 2 and an open condition as shown in FIG. 3 and are biased towards the closed condition by the inherent resilience of the steel of the arms 20, 30. That is to say, the tweezers will apply their own force to continuously grasp an item. The item is released by squeezing the arms of the tweezers together to open the tweezers.

A grasping formation in the form of beaks 22, 32 is provided at the respective ends of each of the arms 20, 30. The beaks have part cylindrical faces which each define one half of a cylinder. In use the cylindrical faces grasp the handle of an endodontic hand file. The axis of the cylindrical faces is offset from a direction perpendicular to the length of the arms of the tweezers by 20 degrees (+/−3 degrees), as indicated by dashed line B in FIG. 1.

In order to grasp a hand file with the tweezers 10 the arms 20, 30 are squeezed together as indicated by arrows D in FIG. 4. This causes the grasping formations to separate indicated by arrows E as the tweezers move to the open configuration. A hand file 100 can then be introduced between the grasping formations.

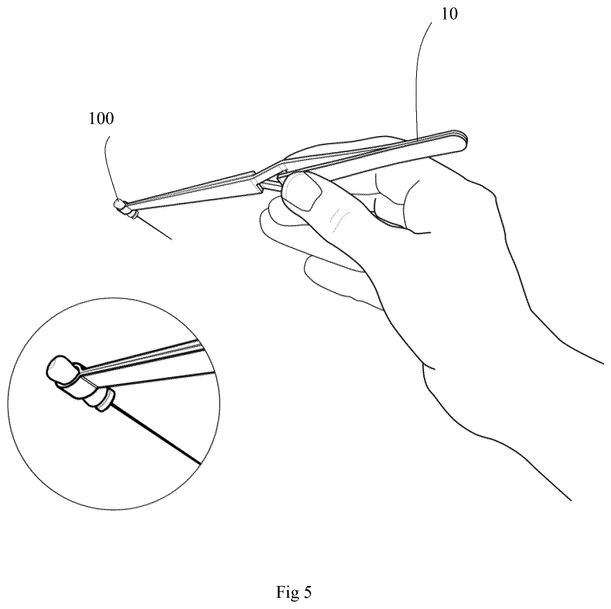

When pressure is released from the arms 20, 30 of the tweezers then the grasping formations move towards one another to grasp the file 100 as shown in FIG. 5.

Tweezer 10 is a narrow reverse action tweezer with a circular shaped tip for holding, placing, securely moving and removing an endodontic hand file in the root canal space inside a tooth.

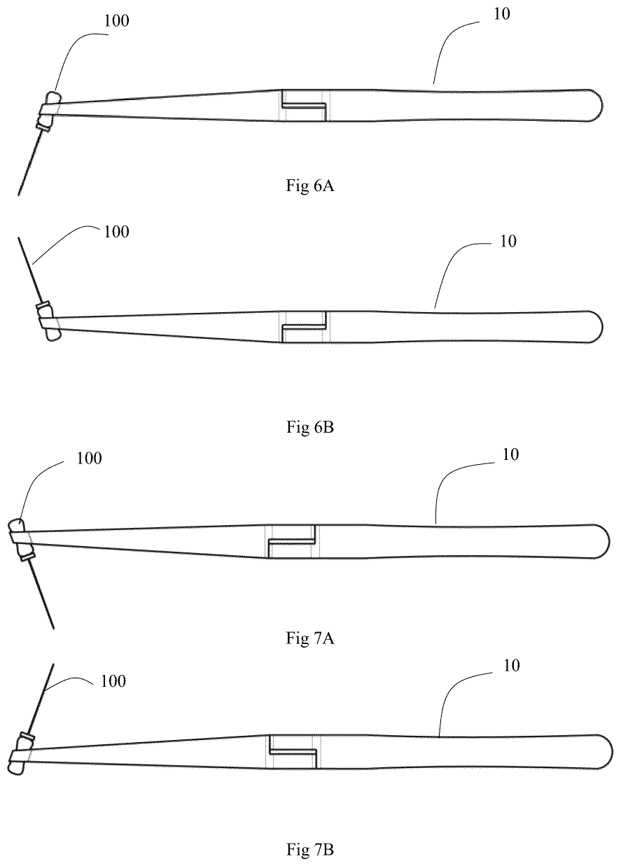

Referring to FIGS. 6A to 7B, when engaged, the file angle is 20 degrees to the line perpendicular to the long axis of the tweezer with bidirectional use on both sides of the tweezer, on opposite sides to the perpendicular as follows:

In FIGS. 6A and 6B the file 100 has been engaged with the file tip pointing outwardly of the end of the tweezers 10. The tweezer and file combination can then be held in a downwardly directed orientation FIG. 6A to work on a lower row of teeth or held in an upwardly directed orientation FIG. 6B to work on an upper row of teeth.

In FIGS. 7A and 7B the file 100 has been engaged with the file tip pointing inwardly of the end of the tweezers 10. The tweezer and file combination can then be held in a downwardly directed orientation FIG. 7A to work on a lower row of teeth or held in an upwardly directed orientation FIG. 7B to work on an upper row of teeth.

Embodiments of the invention have at least the following advantages over lockable forceps:
No locking clasp mechanism. The reverse action assembly does not use any locking clasp mechanism that may be subject to loosening or jamming that would prevent the tweezer from engaging and disengaging the endodontic hand file. No secondary movement of locking or unlocking the clasp to secure or release the endodontic hand file is required.
Strong and secure grasping mechanism. The resting active force is strong enough to secure the endodontic hand file, without slipping or rotation during placement or manoeuvring, whilst being held in the beaks of the tweezers. It allows for instrument exchanging with an assistant without the risk of the endodontic hand file being displaced from the beaks.
Slimline design. The slim profile improves visibility around the endodontic hand file during use by allowing for additional space around the tweezer head. This allows the fingers and hands to be positioned away from the tooth and out of the line of sight of the dentist.

Any reference to prior art contained herein is not to be taken as an admission that the information is common general knowledge, unless otherwise indicated.

Finally, it is to be appreciated that various alterations or additions may be made to the parts previously described without departing from the spirit or ambit of the present invention.

1-9. (canceled)

10. Tweezers for use in conducting root canal therapy including:
a pair of elongate arms;
the arms are moveable between a closed condition and an open condition and are biased towards the closed condition;
a grasping formation is provided at an end of each of the arms;
the grasping formations have substantially part cylindrical faces to grasp opposite sides of the handle of an endodontic file with bidirectional use on both sides of the tweezers;
wherein axes of the cylindrical faces are offset from a direction perpendicular to the length of the arms; and wherein the axes of the cylindrical faces are offset by between 10 and 30 degrees.

11. The tweezers according to claim 10 wherein the axes of the cylindrical faces are offset by between 15 and 25 degrees.

12. The tweezers to claim 10 wherein the axes of the cylindrical faces are offset by between 17 to 23 degrees.

13. A method of conducting root canal therapy including the steps of:
providing tweezers according to claim 10;
grasping an endodontic file using the tweezers; and
using the tweezers and the endodontic file to conduct root canal therapy.

14. A method of conducting root canal therapy including the steps of:
providing tweezers according to claim 11;
grasping an endodontic file using the tweezers; and
using the tweezers and the endodontic file to conduct root canal therapy.

15. A method of conducting root canal therapy including the steps of:
providing tweezers according to claim 12;
grasping an endodontic file using the tweezers; and
using the tweezers and the endodontic file to conduct root canal therapy.
